(19) United States
(12) Patent Application Publication
(10) Pub. No.: US 2015/0322083 A1
(43) Pub. Date: Nov. 12, 2015

(54) (THIENO[2,3-b][1,5]BENZOXAZEPIN-4-YL)PIPERAZIN-1-YL COMPOUNDS AS SELECTIVE 5-HT2A INVERSE AGONISTS

(71) Applicant: Eli Lilly and Company, Indianapolis, IN (US)

(72) Inventor: Andrew James LEDGARD, Berkshire (GB)

(21) Appl. No.: 14/692,780

(22) Filed: Apr. 22, 2015

Related U.S. Application Data
(60) Provisional application No. 61/985,677, filed on Apr. 29, 2014.

(51) Int. Cl. C07D 498/04

(52) U.S. Cl. CPC C07D 498/04

(57) ABSTRACT

A 5-HT_{2A} receptor inverse agonist of the formula:

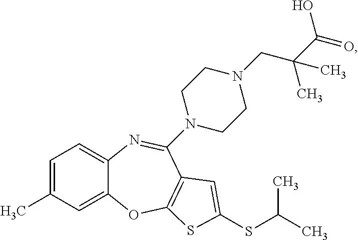 [SMILES: Cc1ccc2c(c1)Oc1sc(SC(C)C)cc1C(N1CCN(CC(C)(C)C(=O)O)CC1)=N2]

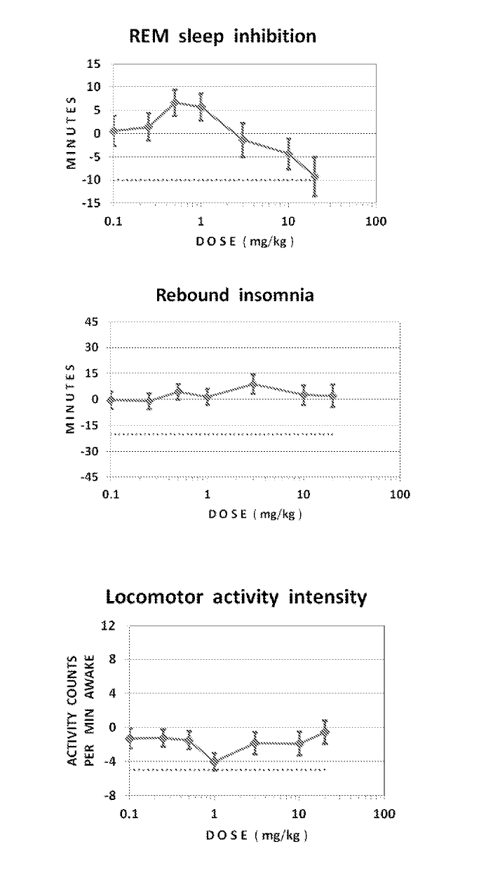

its uses and methods for its preparation are described.

This application claims priority to U.S. provisional application Ser. No. 61/985,677, filed Apr. 29, 2014.

Serotonin plays important roles in a variety of physiological processes through its interaction with at least fourteen different G-protein coupled receptors. Modulation of 5-HT_{2A} receptors in the central nervous system plays a key role in mood regulation, in addition to contributing to the sleep/wake cycle. It is also thought that the 5-HT_{2A} antagonist/inverse agonist component of a number of non-selective/multi-activity antipsychotics plays an important role in remediating psychotic symptoms.

Historically, antipsychotics used to treat schizophrenia have also been utilized to control psychosis and behavioral disorders such as agitation or aggression in Alzheimer's disease and Parkinson's disease. However, side effects with these elderly patient populations now counter-indicate their use for the treatment of behavioral disorders, as evidenced by the U.S. Food & Drug Administration issuing a Public Health Advisory: Deaths with Antipsychotics in Elderly Patients with Behavioral Disturbances (11 Apr. 2005), which calls for black box warnings on atypical antipsychotics, noting increased mortality in elderly patients with dementia (i.e. in Alzheimer's and Parkinson's disease patients among other dementia disorders), and also noting that their use for the treatment of behavioral disorders in elderly patients with dementia has not been approved. There is also evidence that serotonergic pathways are adversely affected in Alzheimer's disease patients and Parkinson's diseases patients, and that a selective 5HT_{2A} antagonist or inverse agonist could alleviate associated psychotic symptoms. (See Price et al., Pimavanserin, a 5-HT_{2A} receptor inverse agonist, reverses psychosis-like behaviors in a rodent model of Alzheimer's disease, Behavioural Pharmacology vol. 23:426-433 (2012); and Cummings et al., Pimavanserin for patients with Parkinson's disease psychosis: a randomized, placebo-controlled phase 3 trial, The Lancet Vol 383:533-540 (2014).)

WO 2007/022068 describes certain substituted (thieno[2,3-b][1,5]benzodiazepine-4-yl)piperazin-1-yl and (thieno[2,3-b][1,5]benzoxazepine-4-yl)piperazin-1-yl compounds as H1 receptor antagonists, selective against muscarinic and adrenergic receptors for treating sleep disorders.

WO 2013/032804 and WO 2013/009517 disclose two substituted (thieno[2,3-b][1,5]benzoxazepine-4-yl)piperazin-1-yl compounds as dual activity H1 receptor inverse agonists/5-HT_{2A} receptor antagonists for the treatment of sleep disorders. The H1 receptor activity in these compounds is deemed important for the sleep on-set component of the overall therapeutic activity of the compounds for the treatment of insomnia

The present invention provides 2,2-dimethyl-3-(4-{8-methyl-2-[(1-methylethyl)sulfanyl]thieno [2,3-b][1,5]benzoxazepin-4-yl}piperazin-1-yl)propionic acid and pharmaceutically acceptable salts thereof. The compounds demonstrate inverse agonist potency for the 5-HT_{2A} receptor, and selectivity as against histamine receptors, other serotonin receptors, and other physiologically relevant receptors, particularly as against the 5-HT_{2C} receptor, GABA_{A} receptor, dopaminergic receptors, adrenergic receptors, and the hERG channel. These compounds demonstrate through animal models that they are brain penetrant inverse agonists of the 5-HT_{2A} receptor, are active in animal models of psychosis, and also promote sleep consolidation without reduction of REM sleep. As a result of these in vitro and in vivo obervations, these compounds are believed to be useful for the treatment of psychosis in Alzheimer's disease patients or Parkinson's disease patients. As a result of these data, the compounds are alternatively believed to be useful for the treatment of behavioral disturbances associated with Alzheimer's disease or Parkinson's disease, as for example aggression or agitation; mood disorders associated with Alzheimer's disease or Parkinson's disease, as for example depression, anxiety or apathy; and/or sleep distrubances, as for example night time wandering.

The present invention provides a compound of Formula I

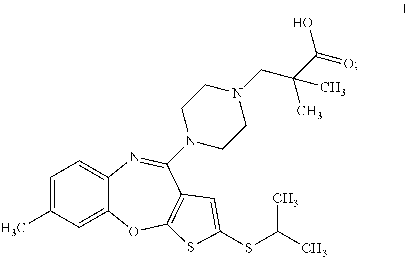 [SMILES: Cc1ccc2c(c1)Oc1sc(SC(C)C)cc1C(N1CCN(CC(C)(C)C(=O)O)CC1)=N2]

or a pharmaceutically acceptable salt thereof; that is to say 2,2-dimethyl-3-(4-{8-methyl-2-[(1 -methylethyl)sulfanyl]thieno[2,3-b][1,5]benzoxazepin-4-yl}piperazin-1-yl)propionic acid, or a pharmaceutically acceptable salt thereof.

In another aspect of the invention there is provided a pharmaceutical composition comprising a compound of Formula I, or a pharmaceutically acceptable salt thereof, and at least one pharmaceutically acceptable carrier, diluent, or excipient. Furthermore, this aspect of the invention provides a pharmaceutical composition for treating psychosis in Alzheimer's disease patients, comprising a compound of Formula I, or a pharmaceutically acceptable salt thereof, and one or more pharmaceutically acceptable excipients, carriers, or diluents. This aspect of the invention also provides a pharmaceutical composition for treating psychosis in Parkinson's disease patients, comprising a compound of Formula I, or a pharmaceutically acceptable salt thereof, and one or more pharmaceutically acceptable excipients, carriers, or diluents. In another embodiment of this aspect of the invention there is provided a pharmaceutical composition for treating agitation and/or aggression in Alzheimer's disease patients, comprising a compound of Formula I, or a pharmaceutically acceptable salt thereof, and one or more pharmaceutically acceptable excipients, carriers, or diluents. In another embodiment of this aspect of the invention there is provided a pharmaceutical composition for treating agitation and/or aggression in Parkinson's disease patients, comprising a compound of Formula I, or a pharmaceutically acceptable salt thereof, and one or more pharmaceutically acceptable excipients, carriers, or diluents.

In another aspect, the invention provides a compound of Formula I or a pharmaceutically acceptable salt thereof for use in therapy. Within this aspect, the invention provides a compound of Formula I, or a pharmaceutically acceptable salt thereof, for use in the treatment of psychotic symptoms and/or behavioral disturbances associated with Alzheimer's disease and/or Parkinson's disease. Psychotic symptoms may include hallucinations and/or delusions. Behavioral disturbances may include mood disorders such as depression, agitation, aggression, apathy, (night-time) wandering and sleep disturbances. As such, the invention provides a compound of Formula I, or a pharmaceutically acceptable salt thereof, for use in the treatment of psychosis associated with Alzheimer's disease. The invention provides a compound of Formula I, or a pharmaceutically acceptable salt thereof, for use in the treatment of psychosis associated with Parkinson's disease. Further, the invention provides a compound of Formula I, or a pharmaceutically acceptable salt thereof, for use in the treatment of agitation or aggression associated with Alzheimer's disease. Yet further, the invention provides a compound of Formula I, or a pharmaceutically acceptable salt thereof, for use in the treatment of agitation or aggression associated with Parkinson's disease. Also within this aspect, the invention provides a compound of Formula I, or a pharmaceutically acceptable salt thereof, for use in the treatment of dementia, as for example dementia associated with fronto-temporal lobar degeneration, Lewy-body dementia, vascular dementia, etc. It will be understood that many of these forms of dementia may present as comorbid with dementia associated with Alzheimer's disease and/or Parkinson's disease and that the invention contemplates the use of the compound of Formula I, or a pharmaceutically acceptable salt thereof, in the treatment of such comorbid dementias.

Another aspect of the invention provides the use of a compound of Formula I, or a pharmaceutically acceptable salt thereof, in the manufacture of a medicament for the treatment of psychotic symptoms and/or behavioral disturbances associated with Alzheimer's disease and/or Parkinson's disease. In another embodiment of this aspect of the invention, there is provided use of a compound of Formula I, or a pharmaceutically acceptable salt thereof, in the manufacture of a medicament for the treatment of psychosis associated with Alzheimer's disease and/or Parkinson's disease. There is further provided use of a compound of Formula I, or a pharmaceutically acceptable salt thereof, in the manufacture of a medicament for the treatment of agitation or aggression associated with Alzheimer's disease and/or Parkinson's disease.

It another aspect, the present invention also provides a method of treating psychotic symptoms and/or behavioral disturbances associated with Alzheimer's disease and/or Parkinson's disease. Psychotic symptoms may include hallucinations and/or delusions. Behavioral disturbances may include mood disorders such as depression, agitation, aggression, apathy, (night-time) wandering and sleep disturbances. In one embodiment, the invention provides a method of treating psychosis associated with Alzheimer's disease, comprising administering to a patient in need of such treatment an effective amount of a compound of Formula I, or a pharmaceutically acceptable salt thereof. In another embodiment, the invention provides a method of treating psychosis associated with Parkinson's disease, comprising administering to a patient in need of such treatment an effective amount of a compound of Formula I, or a pharmaceutically acceptable salt thereof. In yet another embodiment, the invention provides a method of treating agitation or aggression associated with Alzheimer's disease, comprising administering to a patient in need of such treatment an effective amount of a compound of Formula I, or a pharmaceutically acceptable salt thereof. In yet another embodiment, the invention provides a method of treating agitation or aggression associated with Parkinson's disease, comprising administering to a patient in need of such treatment an effective amount of a compound of Formula I, or a pharmaceutically acceptable salt thereof.

For clarity, the following numbering of the tricyclic ring structure will be used throughout the application:

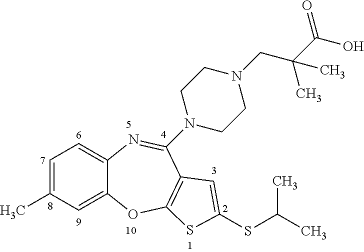 [SMILES: Cc1ccc2c(c1)Oc1sc(SC(C)C)cc1C(N1CCN(CC(C)(C)C(=O)O)CC1)=N2]

The compound of this invention has basic and acidic moieties, and accordingly reacts with a number of organic and inorganic acids and bases to form pharmaceutically acceptable salts. Pharmaceutically acceptable salts of the compound of the present invention are contemplated within the scope of the present application. The term “pharmaceutically acceptable salt” as used herein, refers to any salt of a compound of the invention that is substantially non-toxic to living organisms. Such salts include those listed in Journal of Pharmaceutical Science, 66, 2-19 (1977), which are known to the skilled artisan.

For purposes of this application, a patient is a human suffering from any disease or disorder.

For purposes of this application, ‘treat’, ‘treating’, or ‘treated’ in relation to a patient means to manage a disease or disorder by medicinal measures, and ‘treatment’ means the medical management of a patient, as for example by administration of a therapeutic substance intended to ameliorate a basic disease or disorder problem.

For purposes of this application, the term “effective amount” refers to the amount or dose of compound of the invention, or a pharmaceutically acceptable salt thereof which, upon single or multiple dose administration to the patient, provides the desired effect in the patient under diagnosis or treatment.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the patient's size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

Abbreviations used herein are defined as follows:

“5-HT” means 5-hydroxytryptamine, which is serotonin. Subscripted leters after “5-HT”, as in “5-HT_{2A}”, “5-HT_{2B}”, “5-HT_{2C}”, etc., refer to the various subtypes of serotonin receptors found in mammals, each with its distinct sequence, distribution and physiological role.

“ANOVA” means analysis of variance.

“Brine” means saturated aqueous NaCl solution.

“DCM” means dichloromethane.

“DMSO” means dimethyl sulfoxide.

“DOI” means the 5-HT_{2A} receptor agonist 2,5-dimethoxy-4-iodoamphetamine.

“EDTA” means ethylenediaminetetraacetic acid.

“EEG” means electroencephalography.

“EMG” means electromyograph

“EtOAc ” means ethyl acetate.

“Equiv” means equivalent(s).

“FLIPR®” means Fluorometic Imaging Plate Reader and is a registered mark of Molecular Devices, LLC.

“GABA” means γ-aminobutyric acid.

“hERG” means human ether a-gogo related gene.

“HPLC” means high pressure liquid chromatography.

“hr.” means hours.
“IC₅₀” means the concentration at which 50% of the maximum inhibition is achieved.

“LC-MS” means HPLC-mass spectrography.

“LMI” means locomotor intensity.

“MeOH” means methanol.

“min. ” means minutes.

“MS” means mass spectroscopy.

“MS (ES+)” means mass spectroscopy using electrospray ionization.

“NMDA” means N-methyl-D-aspartic acid

“NMR” means nuclear magnetic resonance.

“Pd₂dba₃” means tris(dibenzylideneacetone)dipalladium(0)

“PO” means per os

“REM” means rapid eye movement.

“RO” means receptor occupancy

“SEM” means standard error of the mean.

“THF” means tetrahydrofuran.

“VGCCs” means voltage gated calcium channels.

### General Chemistry

The compound of the present invention can be prepared according to the following synthetic examples.

### Preparation 1: 2,5-Dibromo-thiophene-3-carboxylic acid

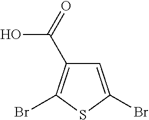

To a solution of 3-thiophenecarboxylic acid (92.39 g; 720.94 mmoles) in acetic acid (500.00 mL; 524.00 g) at 85° C., add bromine (2.02 moles; 103.73 mL; 322.59 g, in acetic acid (125.00 mL; 131.00 g)) drop wise in 2 portions over 100 min. Stir the mixture at 85° C. for 135 min. Pour the briefly cooled mixture into a 5 L conical flask containing water (2.50 L) stirring with an overhead stirrer for 15 min. Filter and wash the solid cake with water until the filtrate runs clear. Air dry the solid. Suspend the solid in water (1.50 L) with stirring at 80° C., add MeOH (50 ml×6), stir at 80° C. for 1 hr., and then cool to ambient temperature overnight. Filter the solid, thoroughly air dry, and then pump to a constant weight in vacuo at 40° C. to yield the title intermediate as an off-white solid (199.2 g, 96.6%); MS m/z 286.7 (M+1).

### Preparation 2: 2,5-Dibromo-N-(2-hydroxy-4-methyl-phenyl)thiophene-3-carboxamide

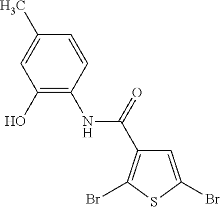 [SMILES: Cc1ccc(NC(=O)c2cc(Br)sc2Br)c(O)c1]

To a suspension of 2,5-dibromo-thiophene-3-carboxylic acid (298.3 g; 1.04 moles) in DCM (1.99 L; 2.63 kg), add dimethylformamide (38.80 mmoles; 3.00 mL; 2.84 g), followed by oxalyl chloride (1.15 moles; 99.56 mL; 145.65 g) in anhydrous DCM (450 mL) over 45 min. Stir the mixture for 150 min., concentrate in vacuo, dissolve the residue in THF (500 mL), and concentrate to give the crude acid chloride. To a mixture of 6-amino-m-cresol (1.36 moles; 170.43 g), pyridine (253.08 mL; 247.56 g) and THF (2.24 L; 1.98 kg) add the crude acid chloride dissolved in THF (993.34 mL; 880.20 g) over about 50 min., keeping the internal temperature at about 10° C. Upon complete addition, allow the mixture to come to ambient temperature and stir for 1 hr. Pour the resultant suspension into a mixture of 2 M HCl (3.00 L) and ice (1.00 L) with overhead stirring to give a beige suspension. Filter the suspension and sequentially wash the cake with 2 M HCl (1.00 L) and then water (1.00 L), and then thoroughly dry the solid before drying in vacuo over P₂O₅. Yield (351.8 g, 86.2%); MS m/z 391.9 [M+1].

### Preparation 3: 2-Bromo-8-methyl-5H-thieno[2,3-b][1,5]benzoxazepin-4-one

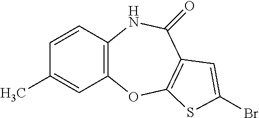 [SMILES: Cc1ccc2c(c1)Oc1sc(Br)cc1C(=O)N2]

Charge a reactor with 2,5-dibromo-N-(2-hydroxy-4-methyl-phenyl)thiophene-3-carboxamide (200 g; 511.40 mmoles), followed by DMSO (4.00 L), with stirring at ambient temperature until dissolved. Add potassium carbonate (613.68 mmoles; 84.81 g) and heat overnight with a heater jacket set to 90° C. Increase the jacket temperature to 95° C. for 1 hr. Reset the temperature to 40° C. and dilute the mixture with water (4.00 L) at a rate such that the internal temperature does not rise above 55° C. Stir the mixture for 30 min. and then filter into two equal portions through large sintered funnels. Wash each bed with water (1×1.50 L), MeOH (2×1.00 L), and finally diethyl ether (1×1.00 L) and then thoroughly dry. The two batches are combined and ground up with a mortar and pestle to give a free flowing grey powder that is dried to constant weight in vacuo over P₂O₅. Yield (114.6 g, 72.3%); MS m/z 311.9 [M+1].

### Preparation 4: 2-Bromo-4-chloro-8-methyl-thieno[2,3-b][1,5]benzoxazepine

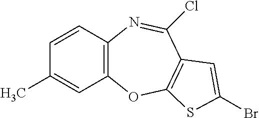 [SMILES: Cc1ccc2c(c1)Oc1sc(Br)cc1C(Cl)=N2]

Add N,N-dimethylaniline (451.36 mmoles; 57.29 mL) to a suspension of 2-bromo-8-methyl-5H-thieno[2,3-b][1,5]benzoxazepin-4-one (50 g; 161.20 mmoles) in methoxybenzene (250.00 mL; 248.20 g) and heat to 80° C. to give a thick muddy mass. Add phosphoryl chloride (370.76 mmoles; 34.45 mL) drop-wise and heat at 100° C. with stirring for 2 hr., during which the gelatinous looking reaction mixture begins to stir more freely to give first, a muddy brown mixture, and finally a dark solution. The mixture is cooled to ambient temperature and concentrated on a rotary evaporator with the aid of a trolley pump to give a dark residue. Treat the residue twice with anhydrous toluene (250 mL) and after each addition the mixture is rotated to give a dark solution which again is concentrated in vacuo. Isolate a thick dark brown mass and assume a quantitative yield. (52.97 g, 99.9%); MS m/z 329.9 [M+1].

### Preparation 5: Methyl 2,2-dimethyl-3-oxo-propanoate

Add methyl 3-hydroxy-2,2-dimethylpropanoate (33 g, 250 mmol) to Dess-Martin periodinane (106 g, 250 mmol) suspended in DCM (1.00 L) at 0° C. and stir at room temperature for 18 hr. Filter the reaction mixture through a CELITE® bed and concentrate the filtrate. Wash the concentrated filtrate with pentane (2×200 mL). Separate the pentane layer and concentrate in vacuo to give methyl 2,2-dimethyl-3-oxo-propanoate (31.93 g, quantitative). ¹H NMR (d₆-DMSO) δ 9.59 (s, 1H), 3.67 (s, 3H), 1.26 (s, 6H).

### Preparation 6: Methyl 3-(4-((1,1-dimethylethoxy)carbonyl)piperazin-1-yl)-2,2-dimethylpropanoate

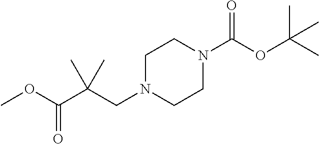 [SMILES: COC(=O)C(C)(C)CN1CCN(C(=O)OC(C)(C)C)CC1]

Stir a solution of methyl 2,2-dimethyl-3-oxo-propanoate (30.88 g, 237.31 mmoles) and piperazine-1-carboxylic acid tert-butyl ester (34.00 g, 182.55 mmoles) in DCM (500 mL) at room temperature for 20 min. Add acetic acid (2 equiv); 20.92 mL, 365.09 mmoles) followed by sodium triacetoxyborohydride (1.4 equiv; 54.17 g, 255.56 mmoles) over 0.5 hr. and stir the resulting mixture at room temperature overnight. Carefully quench with water (250 mL) and transfer the mixture to a separating funnel with DCM (300 mL). Wash the resulting organic layer with brine. Dry over MgSO₄, filter and evaporate to give methyl 3-(4-((1,1-dimethylethoxy)carbonyl)piperazin-1-yl)-2,2-dimethylpropanoate (58 g, 100%). MS (m/z): 301.2 [M+1].

### Preparation 7: Methyl 2,2-dimethyl-3-piperazin-1-ylpropanoate dihydrochloride

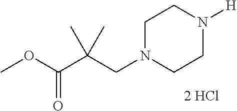

To a solution of methyl 3-(4-(1,1-dimethylethoxy)carbonyl)piperazin-1-yl)-2,2-dimethylpropanoate (58.0 g, 193.08 mmoles) in isopropyl alcohol (150 mL) add a 4 M dioxane solution of HCl ((4 equiv); 193.08 mL, 772.31 mmoles) over 15 min., observing gas evolution and a fine precipitate. Heat at 55° C. for 3 hr. to give a white precipitate. Cool to 10° C. and collect the white solid by filtration. Wash with further isopropyl alcohol (30 mL), and then wash with EtOAc. Dry in a vacuum oven at 45° C. for 1 hr. to give methyl 2,2-dimethyl-3-piperazin-1-ylpropanoate dihydrochloride (31 g, 59% yield). MS (m/z): =201.1 [M+1].

### Preparation 8: Methyl 3-[4-(2-bromo-8-methyl-4,5-dihydrothieno[2,3-b][1,5]benzoxazepin-4-yl)piperazin-1-yl]-2,2-dimethyl-propanoate

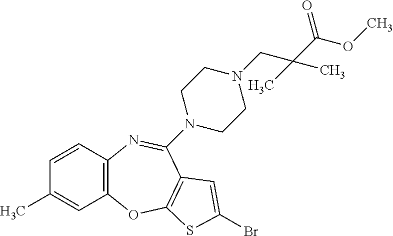

Slurry 2-bromo-4-chloro-8-methyl-thieno[2,3-b][1,5]benzoxazepine (52.97 g, 161.19 mmoles) in anhydrous acetonitrile (600 mL; 11.45 moles; 600 mL; 469.98 g) and add methyl 2,2-dimethyl-3-piperazin-1-ylpropanoate dihydrochloride (322.38 mmoles; 88.08 g) to give a suspension. Add potassium carbonate (822.08 mmoles; 113.62 g) and reflux overnight with stirring. Hot filter the mixture, wash the beige solid bed with copious amounts of acetonitrile. Concentrate the dark filtrate in vacuo to give a brown oily solid. Add MeOH (200 ml), stir for 10 min. and then filter. Wash the bed, with stirring, with MeOH (2×100 ml), followed by iso-hexane (100 ml), and then air dry followed with drying in vacuo over night at 50° C. to provide crude product, (yield 68.5 g, 68.1%).

Combine an additional three batches of crude methyl 3-[4-(2-bromo-8-methyl-4,5-dihydrothieno[2,3-b][1,5]benzoxazepin-4-yl)piperazin-1-yl]-2,2-dimethyl-propanoate prepared essentially as above, with the above material (135.7 g total) and proceed to further purify as follows: Suspend the combined lots in DCM (200 mL) and load onto a silica gel plug (1 kg,) pre-equilibrated in DCM-isohexane (1:1). Elute with DCM-isohexane (1:1) to remove any residual anisole. Finally elute the product with EtOAc:isohexane (1:9), combine all the product fractions and concentrate under reduced pressure to provide a lemon colored solid, pump to constant weight in a vacuum oven at 40° C. Yield (97.96 g, 88.9%), MS m/z 494.0 [M+1].

### Preparation 9: Methyl 3-[4-(2-isopropylsulfanyl-8-methyl-thieno[2,3-b][1,5]benzoxazepin-4-yl)piperazin-1-yl]-2,2-dimethyl-propanoate

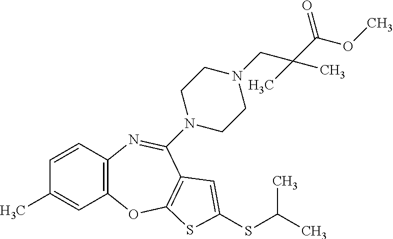

Charge a reactor with methyl 3-[4-(2-bromo-8-methyl-thieno[2,3-b][1,5]benzoxazepin-4-yl)piperazin-1-yl]-2,2-dimethyl-propanoate (126.7 g; 257.29 mmoles) in THF (1.14 L; 1.01 kg) and diisopropylethylamine (643.23 mmoles; 112.18 mL). Degas the mixture with a nitrogen purge for 15 min. Add 2-propanethiol (283.02 mmoles; 26.43 mL), Pd₂dba₃ (0.005 equiv (molar); 1.29 mmoles; 1.18 g) and xantphos (4,5-Bis(diphenylphosphino)-9,9-dimethylxanthene) (0.005 equiv (molar); 1.29 mmoles; 744.39 mg) and heat at a gentle reflux for 1 hr. Cool the mixture to ambient temperature and then concentrate in vacuo to give a dark solid. Partition the solid between EtOAc (2.50 L) and water (2.50 L) with rapid stirring and separate. Wash the organic phase with water (1.00 L) followed by saturated brine solution (1.00 L) and dry over magnesium sulfate. Concentrate under reduced pressure to provide a tan colored solid. Dissolve the solid material in DCM (150 mL), dilute with hexane (50 mL) and then load onto a pad of silica (1 Kg, pre-equilibrated in iso-hexane). Elute with iso-hexane to remove a yellow colored impurity, then elute with 5% EtOAc in iso-hexane to remove an orange impurity. Finally, elute with 10%-15% EtOAc in iso-hexane gradient to elute the product. Combine all product fractions and concentrate under reduced pressure to provide a yellow solid. Dry the solid to constant weight in the vacuum oven at 40° C. Yield (116.5 g, 91.2%), MS m/z 488.2 [M+1].

### EXAMPLE 1

### 3-[4-(2-isopropylsulfanyl-8-methyl-thieno[2,3-b][1,5]benzoxazepin-4-yl)piperazin-1-yl]-2,2-dimethyl-propanoic acid

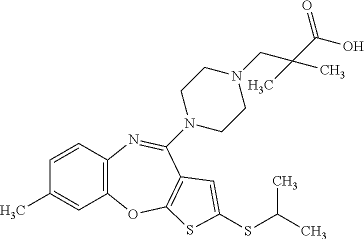 [SMILES: Cc1ccc2c(c1)Oc1sc(SC(C)C)cc1C(N1CCN(CC(C)(C)C(=O)O)CC1)=N2]

Add a solution of NaOH (697.71 mmoles; 27.91 g) in water (725 mL) to a suspension of methyl 3-[4-(2-isopropylsulfanyl-8-methyl-thieno[2,3-b][1,5]benzoxazepin-4-yl)piperazin-1-yl]-2,2-dimethyl-propanoate (115.5 g; 232.57 mmoles) in isopropyl alcohol (725 ml; 569.92 g) and heat the yellow suspension with stirring at 85° C. for 5 hr. Allow the hazy orange mixture to cool to ambient temperature overnight. Cool the reaction in an ice bath and adjust the pH to pH 7 using concentrated HCl, followed by 5 M HCl as the endpoint is approached. Place the yellow milky mixture under reduced pressure to remove the majority of the organics leaving a thick yellow mass. Add EtOAc (1.00 L) and rotary evaporate for 15 min. Redissolve with the addition of EtOAc (500 mL) and water (250 mL) with stirring. Separate the layers, extract the aqueous phase with EtOAc (200 mL), combined the organic phases, wash with water (500 mL) and then brine (500 mL), and then dry over magnesium sulphate. Concentrate the mixture to a yellow foam which is then broken up and pumped to constant weight in vacuo. Dissolve the residue in EtOAc (500 mL) and heat to reflux with stiffing. Add additional EtOAc (100 mL) to give a solution with a very faint haze, dilute with iso-hexane (1.20 L), and allow the mixture to cool to ambient temperature overnight. Filter the precipitated solid and wash with chilled 2:1 iso-hexane:EtOAc (100 mL). Thoroughly air dry the solid and then oven dry under vacuum to constant weight at 40° C. Yield (53.6 g, 48.6%), MS m/z 474.2 [M+1].

### EXAMPLE 2

### 3-[4-(2-Isopropylsulfanyl-8-methyl-thieno[2,3-b][1,5]benzoxazepin-4-yl)piperazin-1-yl]-2,2-dimethyl-propanoic acid dihydrochloric acid salt

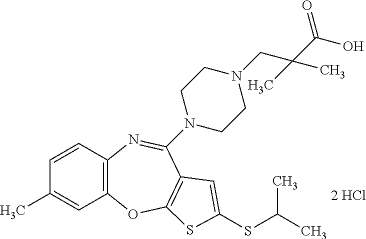 [SMILES: Cc1ccc2c(c1)Oc1sc(SC(C)C)cc1C(N1CCN(CC(C)(C)C(=O)O)CC1)=N2]

Slurry 3-[4-(2-isopropylsulfanyl-8-methyl-thieno[2,3-b][1,5]benzoxazepin-4-yl)piperazin-1-yl]-2,2-dimethyl-propanoic acid (5.53 g, 11.68 mM) in acetonitrile (170 mL, 3.24 M,) and acidify with 2 M HCl (23.35 mL, 4 eqs.). Stir the yellow solution at room temperature for 1 hr. and then evaporate the mixture to dryness under reduced pressure. Add acetonitrile and water, evaporate under reduced pressure at 40° C., and then oven dry under vacuum to constant weight at 40° C. to give an off-white solid. Yield (6.40 g, 100.29%.), MS m/z 474.00, [MH+].

### EXAMPLE 3

### 3-[4-(2-isopropylsulfanyl-8-methyl-thieno[2,3-b][1,5]benzoxazepin-4-yl)piperazin-1-yl]-2,2-dimethyl-propanoic acid mono-hydrochloride salt

Slurry 3-[4-(2-isopropylsulfanyl-8-methyl-thieno[2,3-b][1,5]benzoxazepin-4-yl)piperazin-1-yl]-2,2-dimethyl-propanoic acid (155 mg) in 1.50 mL of acetone. Heat at 60° C. with stiffing (800 rpm) to dissolve to a clear, light yellow solution. Add 750 μL HCl (1 M in EtOAc). Continue stirring the resulting slurry at 600 rpm/60° C. for 15 min. and then allow the slurry to cool to room temperature for 1 hr. without stirring to provide a thick slurry of white solid. The bright white solid was isolated by vacuum filtration and dried under continued vacuum with an air stream over the cake (142 mg, 85% yield).

### EXAMPLE 4

### 3-[4-(2-isopropylsulfanyl-8-methyl-thieno[2,3-b][1,5]benzoxazepin-4-yl)piperazin-1-yl]-2,2-dimethyl-propanoic acid phosphate salt

Slurry 3-[4-(2-isopropylsulfanyl-8-methyl-thieno[2,3-b][1,5]benzoxazepin-4-yl)piperazin-1-yl]-2,2-dimethyl-propanoic acid (305 mg) in 6 mL ethanol. Heat the mixture at 70° C. with stiffing (1000 rpm) to dissolve. Add 100 μL phosphoric acid (in 3.90 mL ethanol) and allow the solution to cool to room temperature. When the mixture reaches room temperature, further cool the mixture at 5° C. for 1 hr. to provide a thick layer of bright white solid precipitate under a very light yellowish supernatant. Isolate the white solid by vacuum filtration and dry the resulting cake for 15 mM under continued vacuum with an air stream over the cake. Transfer the solid to a vial and further dry at 70° C. in a vacuum oven overnight to fully dry (328 mg, 89.10% yield).

Literature data (e.g. Price et al., Pimavanserin, a 5-HT_{2A} receptor inverse agonist, reverses psychosis-like behaviors in a rodent model of Alzheimer's disease, Behavioural Pharmacology vol. 23:426-433 (2012); and Cummings et al., Pimavanserin for patients with Parkinson's disease psychosis: a randomized, placebo-controlled phase 3 trial, The Lancet Vol 383:533-540 (2014)) and data generated in non-clinical animal studies support a role for selective 5-HT_{2A} antagonists or inverse agonists in the treatment of psychotic symptoms and/or behavioral disturbances associated with Alzheimer's disease and/or Parkinson's disease. Specifically it is found that the compounds of the invention are selective 5-HT_{2A} inverse agonists, are effective in blocking DOI induced headshaking in mice, are effective in blocking MK-801 induced locomotor hyperactivity in mice, and are effective in increasing total sleep time using EEG monitored rodents without disproportionate or clinically relevant decrease in REM sleep or hypersomnolence.

To further demonstrate the characteristics of the present compounds, they may be run in the following in vitro and in vivo assays:

### In Vitro Binding and Activity Assays:

### 5-HT_{2A} Competition Binding Assay

[³H]-Ketanserin binding experiments are carried out in SPA 96-well format. Membranes used in this assay are prepared from AV-12 cells stably expressing recombinant 5-HT_{2A} receptors (human). The incubation is initiated by the addition of a mixture of WGA YSi SPA beads (1 mg/well, Perkin Elmer (MA, USA), Product No. RPNQ0011) and 2 μg membranes to assay buffer (67 mM Tris, 0.5 mM EDTA; pH 7.6) containing 3.1 nM [³H]-Ketanserin and varying concentrations of the test compound (10 point concentration response curves). Non-specific binding is determined in the presence of 20 μM 1-(1-naphthyl)piperazine. Samples are incubated for 4 hr. at room temperature (22° C.) and then read in a microplate scintillation counter.

### 5-HT_{2C} Competition Binding Assay

[¹²⁵I]-(±)DOI binding experiments are carried out in SPA 96-well format. Membranes used in this assay are prepared from AV-12 cells stably expressing recombinant 5-HT_{2C} receptors (human). The incubation is initiated by the addition of a mixture of WGA PVT SPA beads (0.5 mg/well, Perkin Elmer (MA, USA), RPNQ0001) and 2.5 μg membranes to assay buffer (50 mM Tris-HCl, 10 mM MgCl₂, 0.5 mM EDTA, 10 μM pargyline, 0.1% ascorbic acid, pH7.4) containing 0.2 nM [[¹²⁵I]-(±)DOI and varying concentrations of the test compound (10 point concentration response curves). Non-specific binding is determined in the presence of 20 μM 1-(1-naphthyl)piperazine. Samples are incubated for 4 hr. at room temperature (22° C.) and then read in a microplate scintillation counter.

### Binding Data Analysis

Curves are evaluated using a 4-parameter logistic nonlinear equation to obtain the concentration of competitor causing 50% inhibition of radioligand binding (IC₅₀). Equilibrium dissociation constants (Kᵢ) are calculated according to the equation Kᵢ=IC₅₀/(1+L/K_{d}), where L equals the concentration of radioligand used in the experiment and K_{d} equals the equilibrium dissociation constant of the radioligand for the receptor, determined from standard saturation analysis or homologous competition experiments. Reported values for Kᵢ, where n values are indicated, are shown as geometric mean±the standard error of the mean (SEM), with the number of replicate determinations indicated by n. Geometric means are calculated by the equation GeoMean=10̂(Average(log Kᵢ 1+log Kᵢ 2+ . . . log Kᵢ n)/sqrt n).

### GABA_{A} Antagonism Using Native Receptors in Primary Neuronal Cultures

Activity of compounds on native GABA_{A} receptors is evaluated by monitoring calcium fluxes using a 96 well format FLIPR® system (Fluorometric Imaging Plate Reader (FLIPR®, Molecular Devices). Briefly, cortical embryonic neurons are dissociated from E18 rat embryos and plated at optimum density into black-walled, transparent bottom poly-D-lysine coated 96-well FLIPR® plates. After loading the cells with a calcium sensitive dye (Fluo4-AM, Molecular Devices), the cells are bathed in a solution containing low chloride (chloride replaced by gluconate). Under these conditions activation of GABA_{A} receptors causes an efflux of chloride ions (in the direction of the chemical gradient), which results in membrane depolarization and consequently activation of voltage gated calcium channels (VGCCs). Calcium influx through VGCCs is recorded and analysed offline using the FLIPR® system. For a pharmacological validation of the assay, concentration response curves (CRC) are recorded for the standard agonist (GABA) and standard antagonist (Gabazine). Any effects are determined in CRC mode against a fixed concentration of agonist GABA at 10 μM (equivalent to an EC₉₀ GABA response).

### Methods:

The antagonist effects of compounds are quantified using 10-point dose response curves by comparing the peak fluorescent responses to the agonist GABA in the presence and absence of compound. The assay window is defined as the maximal response obtained by GABA at its predetermined EC₉₀ concentration minus the response obtained by a fully inhibiting concentration of gabazine (50 μM). Antagonist effects are calculated as a percent of the assay window. All data are calculated as relative IC₅₀ values using a four-parameter logistic curve fitting program (Prism Graphpad® 3.01). Antagonist potencies for all compounds are compared to gabazine with three replicates in each assay run.

Further, the compounds of the invention may be tested in binding assays and functional activity assays by well known methods for other physiologically important receptors such as, but not limited to, the hERG channel, other serotonin receptors (specifically 5-HT_{1B} receptors, lack of agonist activity at 5-HT_{2B} receptors, 5-HT_{2C}, 5-HT₅, 5-HT₆, and 5-HT₇ receptors), dopaminergic receptors (specifically D1, D2, and D3), H1 receptors, GABA_{A} receptors, adrenergic receptors and monoamine transporters.

The compounds of Examples 1 and 2 are tested in various in vitro assays essentially as described above and are found to have activity profiles as shown in Table 1.

TABLE 1
Selectivity data

| | Example 1 | Example 2 |
|---|---|---|
| 5-HT_{2A} Kᵢ (nM) | 4.84 | 6.20 |
| 5-HT_{2B} Kᵢ (nM) | 763 | 767 |
| 5-HT_{2C} Kᵢ (nM) | 359 | 772 |
| GABA_{A} IC₅₀ (μM) | NT | 41 |
| hERG Channel (μM) | >100 | >100 |
| Dopamine D₁ Kᵢ (nM) | 2640 | 3760 |
| Dopamine D₂ Kᵢ (nM) | >7660 | >3670 |
| Dopamine D₃ Kᵢ (nM) | >5470 | >5880 |
| H1 Kᵢ (nM) | 599 | 893 |
| 5-HT_{1B} Kᵢ (nM) | >9590 | NT |
| 5-HT₅ Kᵢ (nM) | 6710 | NT |
| 5-HT₆ Kᵢ (nM) | 1130 | NT |
| 5-HT₇ Kᵢ (nM) | >2,060 | NT |
| Adrenergic alpha_{1A} Kᵢ (nM) | 4450 | >13400 |
| Adrenergic alpha_{1B} Kᵢ (nM) | >15800 | 10400 |
| Adrenergic alpha_{2A} Kᵢ (nM) | >1610 | >8820 |
| Adrenergic alpha_{2C} Kᵢ (nM) | >6430 | >6750 |
| Serotonin Transporter Kᵢ (nM) | >521 | >651 |
| Norepinephrine Transport Kᵢ (nM) | >691 | >691 |
| Dopamine Transporter Kᵢ (nM) | >866 | >875 |

NT = Not Tested

Therefore, physiologically relevant doses of the compounds of the invention are expected to provide inhibition of 5-HT_{2A} receptors in vivo, while not interacting with other physiologically relevant receptors to a clinically meaningful extent (i.e. to an extent that would produce a clinically meaningful undesired effect due to activity at that receptor at the minimal doses needed to produce the desired 5-HT_{2A} inhibition), and thus are expected to provide the desired pharmacology while avoiding undesired effects associated with off-target activity. Such undesired effects may include, but are not limited to the following: H1 receptor antagonist activity associated with somnolence/sleep on-set, 5-HT_{2C} antagonist activity associated with treatment emergent weight gain and sleep disturbances, 5-HT_{2B} agonist activity associated with valvulopathy, hERG channel modulation associated with QT prolongation, and GABA_{A} antagonist activity associated with seizure or convulsion activity. Furthermore, it is expected that interference with sleep/wake physiology would be avoided by the selectivity over dopamine receptors, other serotonin receptors, adrenergic receptors, and monoamine transporters.

### 5-HT2A Inverse Agonism:

The inverse agonist activity of compounds of the present invention may be determined by literature recognized assay methods measuring [³⁵S]GTPγS binding to membranes transfected with 5-HT_{2A} receptors in an in vitro activity assay essentially as described below.

Membranes are prepared from frozen pellets of whole AV12 cells expressing the human or rat 5-HT_{2A} receptor. Cells are thawed on ice and homogenized in homogenization buffer (50 mM Tris-HCl, 0.5 mM EDTA, 1 tablet/100 mL of complete EDTA free protease inhibitor tablets (Roche Cat. No. 05 056 489 001), pH=7.4) with 20 strokes of a teflon-glass homogenizer attached to a motorized overhead stirrer. Centrifuge membranes at 1000 rpm (aprox. 150×g) for 15 min. at 4° C. Retain supernatant on ice, resuspend pellets in homogenization buffer, and centrifuge again at 1000 rpm (aprox. 150×g) for 15 min. at 4° C. Add this second supernatant to the first and then centrifuge the combined supernatants for 60 min. at 14,000 rpm (approx. 30,100×g). The final supernatant is discarded and the pellet is resuspended in homogenization buffer and its protein content is determined by the method of Bradford using BSA as the standard. Membrane preparations are stored at −80° C.

Test compound is prepared from a 10 mM DMSO stock solution by serial dilution to provide a 10 point concentration response curve, with a final 1% DMSO concentration when added to the assay mixture. Aliquots of membranes are added to the wells of 96 well assay plates (10 mg membranes/well of 96 well Corning 3604 assay plates, in binding buffer (50 mM Tris-HCl, 100 mM NaCl, 10 mM MgCl₂, 0.2 mM Na-EDTA, 0.135 mM GDP, 13.51 mg/mL saponin). Aliquots of diluted test compound are added to each well and incubated at room temperature for 30 min. [³⁵S]GTPγS (250 μCi; 1250 Ci/mmol, 10 μM Perkin-Elmer NEG030H) is diluted with 10 mM Tricine and 10 mM DTT at pH 7.6 and added to a final concentration of 0.25 nM and membranes are incubated for a further 30 min. at room temperature. The reaction is stopped by adding a mixture of 0.27% Igepal, 0.3125 μ1 Gq Antibody per well and 1.25 mg IgG SPA beads (Perkin-Elmer RPNQ0016)/well. This mixture is incubated for 3 hr. with constant shaking at room temperature, centrifuged at 300 rpm for 1 min. with no brake and incubated for 3 more hr. before reading in a microplate scintillation counter.

The compound of Example 1 is assayed essentially as described and is found to inhibit the intrinsic activity of the recombinant human 5-HT_{2A} receptor with an EC₅₀ value of 0.085 μM (n=2) and that of the recombinant rat 5-HT_{2A} receptor with an EC₅₀ value of 0.103 μM (n=2), thus indicating that the compound is an inverse agonist of the 5-HT_{2A} receptor.

5-HT_{2A} Receptor Occupancy: Receptor occupancy is assayed to demonstrate that the compounds reach and engage the targeted 5-HT_{2A} receptor in vivo. Briefly, male Sprague-Dawley rats (Harlan Sprague-Dawley, Indianapolis, Ind.) weighing approximately 230-280 g are given ad lib access to food and water until the beginning of the 3 hr. experimental protocol. 1 mg/Kg ketanserin (non-selective 5-HT_{2A} antagonist) is used as a positive control to establish assay validity. Test compounds or control are administered by oral gavage in a vehicle comprised of 20% hydroxypropyl beta-cyclodextrin. MDL 100907 ((R)-(+)-α-(2,3-Dimethoxyphenyl)-1-[2-(4-fluorophenyl)ethyl]-4-piperidinemethanol), a selective 5-HT_{2A} antagonist, is used as a tracer. MDL 100907 is suspended in water with 5 μl dilute lactic acid (1 mg/mL), diluted to 6 μg/mL with saline, and administered in a volume of 1 mL/kg intravenously via the lateral tail vein to yield a tracer dose of 3 μg/kg. Rats are administered test compound, ketanserin, or vehicle (N=4), followed 1 hr. later with an intravenous, 3 μg/kg tracer dose of MDL 100907. It is at the time of tracer administration that receptor occupancy (RO) is considered to be measured. Fifteen min. after tracer administration, rats are sacrificed by cervical dislocation. Plasma samples are collected and samples of the frontal cortex and cerebellum are removed. The level of MDL 100907 tracer is measured in each cortical and cerebellar sample. RO is calculated using the well-established ratio method which employs a region of high receptor density representative of total binding (frontal cortex) normalized by an area without or with very low levels of receptor (cerebellum). This region, referred to as the null region, represents nonspecific binding of the ligand probe. Vehicle ratio of the tracer levels in cortex relative to cerebellum represents 0% occupancy. A ratio of 1 represents 100% occupancy and is achieved when all specific binding to the 5-HT_{2A} receptor of the MDL 100907 tracer is blocked. The intermediate ratios of cortical to cerebellar tracer from the test compound pretreated group are interpolated linearly between the ratio of tracer levels in the vehicle-treated animals (0% occupancy) and a ratio of 1 (100% occupancy) in order to determine the percent 5-HT_{2A} RO.

MDL 100907 Analysis: Cortex and cerebellar samples are weighed and placed in conical centrifuge tubes on ice. Four volumes (w/v) of acetonitrile containing 0.1% formic acid is added to each tube. The samples are then homogenized and centrifuged at 14,000 RPM (21,920×g) for 16 min. Supernatant is diluted by adding 100-900 μL sterile water in HPLC injection vials for LC/MS/MS analysis. Analysis of MDL 100907 is carried out using an Agilent model 1200 HPLC (Agilent Technologies, Palo Alto, Calif.) and an API 4000 mass spectrometer. The chromatographic separation is on a 2.1×50 mm C18 column (Agilent part number 971700-907) with a mobile phase consisting of 60% acetonitrile in water with an overall 0.1% formic acid content. Detection of MDL 100907 is accomplished by monitoring the precursor to product ion transition with a mass to charge ratio (m/z) of 374.2 to 123.0. Standards are prepared by adding known quantities of analyte to brain tissue samples from non-treated rats and processing as described above.+−

Statistical Methods: Curves for each study are fitted to a 4 parameter logistic function with the bottom fixed at 0% using JMP® version 8.0 (SAS Institute Inc, Cary N.C.) and the absolute ED₅₀ is calculated by the software. Values are given as means, standard errors and 95% confidence intervals. The compound of Example 1 is tested essentially as described and is found to have 5-HT_{2A} receptor occupancy in vivo with an ED₅₀ of 0.54 mg/kg.

Inhibition of DOI Induced Headshake Activity: The in vivo 5-HT_{2A} receptor antagonist activity of the compounds of the present invention may be further demonstrated by their ability to block head shaking activity induced by the 5-HT_{2A} receptor agonist 2,5-dimethoxy-4-iodoamphetamine (DOI). (see for example Bartoszyk G D, van Amsterdam C, Böttcher H, Seyfried C A. EMD 281014, a new selective serotonin 5-HT_{2A} receptor antagonist. Eur J Pharmacol. 2003 473: 229-230.) This assay has also be used as an indicator of anti-psychotic therapeutic potential. Briefly, male C57BL/6J mice (20-25 g, Charles River) are housed in standard housing conditions (32 mice in a large IVC cage, 07.00 to 19.00 light phase, constant temperature (19-23° C.) and humidity (50%+/−10), ad lib food and water). Mice received either vehicle (0.25% Methyl cellulose), DOI (3 mg/kg in saline) or test compound at doses up to 10 mg/kg PO plus DOI (3 mg/kg in saline). Test compounds are individually evaluated at 4 doses with n=4 for each dose, together with vehicle and DOI+vehicle (n=8). After a test compound pre-treatment time of 60 min. the mice receive either vehicle (saline) or 3 mg/kg DOI dosed subcutaneously, and are then placed into clear perspex observation chambers. Five min. after DOI or vehicle administration the number of visually scored head shakes exhibited by each individual mouse is counted for 15 min. The data is analyzed using an ANOVA and post-hoc Dunnet's Test. The compounds of Example 1 and Example 2 are tested essentially as described and are found to inhibit the DOI induced headshake response with an approximate EC₅₀ of 0.5 mg/kg for both compounds. These results may be deemed as indicative of therapeutic potential as anti-psychotic agenta and 5-HT_{2A} inhibitors (i.e. inverse agonists or antagonists, in that this assay does not distinguish between the two activities).

Sleep and behavioral monitoring in rats: The compounds of the present invention may be tested in rats for their ability to increase the amount of sleep or decrease sleep interruption or both without undesired effects such as inhibition of REM sleep, waking motor impairment, and/or rebound insomnia. Test animals are continuously monitored by electro-encephalograms (EEG), electromyograms (EMG), and motion to measure cumulative nonREM sleep, cumulative total sleep, average sleep bout duration, longest sleep bout duration, rebound insomnia, REM sleep inhibition and locomotor activity intensity during wakefulness. Methods for such studies are known in the art (see for example methods described in Edgar D M, Seidel W F. Modafinil induces wakefulness without intensifying motor activity or subsequent rebound hypersomnolence in the rat. J Pharmacology & Experimental Therapeutics 1997; 283: 757-769; van Gelder R N, Edgar D M, Dement W C. Real-time automated sleep scoring: validation of a microcomputer-based system for mice. Sleep 1991, 14: 48-55; and Gross B A, Walsh C M, Turakhia A A, Booth V, Mashour G A, Poe G R. Open-source logic-based automated sleep scoring software using electrophysiological recordings in rats. J Neurosci Methods. 2009; 184(1):10-8.) Studies are conducted as follows:

Animal preparation. Adult, male Wistar rats (approximately 270-300 g at time of surgery) are surgically fitted for chronic recording of EEG, EMG, and motion as follows: Rats are surgically prepared with a cranial implant consisting of four stainless steel screws for EEG recording (two frontal [3.9 mm anterior from bregma, and ±2.0 mm mediolaterally] and two occipital [6.4 mm posterior from bregma, ±5.5 mm mediolaterally]), and with two Teflon-coated stainless steel wires for EMG recording (positioned under the nuchal trapezoid muscles). All leads are soldered to a miniature connector (Microtech, Boothwyn, Pa.) prior to surgery. The implant assembly is affixed to the skull by the combination of the stainless steel EEG recording screws, cyanoacrylate applied between the implant connector and skull, and dental acrylic. Locomotor activity is monitored via a miniature transmitter (Minimitter PDT4000G, Philips Respironics, Bend, Oreg.) surgically placed into the abdomen. At least 3 weeks are allowed for recovery.

Recording environment. Each rat is housed individually within a microisolator cage modified with an inserted polycarbonate filter-top riser to allow more vertical headroom. A flexible cable that minimally restricts movement is connected at one end to a commutator afixed to the cage top and at the other end to the animal's cranial implant. Each cage is located within separate, ventilated compartments of a stainless steel sleep-wake recording chamber. Food and water are available ad libitum and the ambient temperature is maintained at about 23±1° C. A 24-hr light-dark cycle (LD 12:12) using fluorescent light is maintained throughout the study. Relative humidity averages approximately 50%. Animals are undisturbed for at least 30 hrs before and after each treatment.

Study design and dosing. The vehicle (placebo, methylcellulose 15 centipoise 0.25% in water) or one of the test compound dose levels is administered orally at 1 mL/kg pseudo-randomly such that no rat receives the same treatment twice, and no rat receives more than two of the 8 treatments in any one study. Each rat is removed from its cage for about a minute to be weighed and treated. At least 6 days “washout” period precede and follow each treatment.

Data collection. Sleep and wakefulness discrimination may be automated (e.g., Van Gelder et al. 1991 (above); Edgar et al. 1997 (above); Winrow C J, et al., Neuropharmacology 2010; 58(1):185-94; and Gross et al., 2009 (above). EEG is amplified and filtered (×10,000, bandpass 1-30 Hz), EMG is amplified and integrated (bandpass 10-100 Hz, RMS integration), and non-specific locomotor activity (LMA) is monitored simultaneously. Arousal states are classified in 10 second epochs as non-REM sleep, REM sleep, wakefulness, or theta-dominated wakefulness. Locomotor activity (LMA) is recorded as counts per minute and is detected by commerically available telemetry receivers (ER4000, Minimitter, Bend, Oreg.). Locomotor activity intensity (LMI) is the LMA counts per minute of EEG-defined wakefulness, typically averaged across a desired time period relative to treatment.

Statistical Analysis. All animals having at least one outcome are included in the summary results (for example, we include appropriate data from an animal treatment for which telemetry data is usable but EEG data is not). The post-treatment observation period is divided into post-dosing intervals appropriate to each Outcome, where the time of dosing is defined as the start of Hour=0, and outcomes are summarized in the observation period by computing either the mean hourly or the cumulative value across each period (see legend of Table 2 for precise definition of each Outcome). Sleep bouts are analyzed on the log scale to stabilize the variation, all other variates are analyzed on the linear scale. Each outcome in each period is analyzed by analysis of covariance using treatment group and treatment date as factors and the corresponding pre-treatment interval, 24 hrs earlier, as the covariate. Adjusted means and the change from vehicle means and their corresponding standard errors are summarized for each treatment group. Outcomes analyzed on the log scale are back-transformed to report geometric means and mean ratio-to-vehicle results.

The compound of Example 1 is tested essentially as described. The compound of Example 1 is found to have a statistically significant increase in cumulative NREM sleep time and cumulative total sleep time without statistically significant rebound insomnia or REM sleep inhibition at up to 10 times the conservative efficacy dose. Locomotor activity intensity (LMI), a measure of presumed locomotor activity impairment, however, cannot be ruled out by these data. (See sleep profile and locomotor activity intensity in Table 2.)

TABLE 2
Compound of Example 1.

| | Efficacy variables | | | Undesired effect variables | | |
|---|---|---|---|---|---|---|
| Dose | Cumulative NREM sleep | | | Rebound Insomnia | | |
| (mg/kg PO) | N | Adj. Mean | SE | N | Adj. Mean | LCL |
| 0.1 | 8 | 14.6 | 6.3 | 8 | −0.4 | −10.5 |
| 0.25 | 9 | 17.8 | 5.8 | 9 | −1.0 | −10.1 |
| 0.5 | 9 | 24.1 | 5.9 | 9 | 4.4 | −4.6 |
| 1 | 9 | 30.0 | 5.7 | 9 | 1.5 | −7.6 |
| 3 | 6 | 31.5 | 6.9 | 6 | 8.8 | −2.4 |
| 10 | 6 | 38.3 | 7.6 | 6 | 2.5 | −8.9 |
| 20 | 4 | 40.2 | 7.8 | 4 | 2.1 | −10.7 |
| Dose | Cumulative Total sleep | | | REM inhibition | | |
| (mg/kg PO) | N | Adj. Mean | SE | N | Adj. Mean | LCL |
| 0.1 | 8 | 13.1 | 7.0 | 8 | 0.5 | −5.9 |
| 0.25 | 9 | 21.1 | 6.5 | 9 | 1.4 | −4.5 |
| 0.5 | 9 | 28.2 | 6.5 | 9 | 6.6 | 0.9 |
| 1 | 9 | 31.4 | 6.4 | 9 | 5.6 | −0.1 |
| 3 | 6 | 32.8 | 7.7 | 6 | −1.4 | −8.8 |
| 10 | 6 | 39.1 | 8.5 | 6 | −4.4 | −11.2 |
| 20 | 4 | 38.0 | 8.7 | 4 | −9.3 | −17.6 |
| Dose | Average Sleep Bout | | | Locomotor Activity Intensity | | |
| (mg/kg PO) | N | Adj. Mean | SE | N | Adj. Mean | LCL |
| 0.1 | 8 | 1.09 | 0.11 | 6 | −1.31 | −3.65 |
| 0.25 | 9 | 1.32 | 0.13 | 8 | −1.22 | −3.27 |
| 0.5 | 9 | 1.75 | 0.17 | 7 | −1.49 | −3.62 |
| 1 | 9 | 1.71 | 0.16 | 8 | −4.04 | −6.10 |
| 3 | 6 | 1.75 | 0.20 | 5 | −1.86 | −4.42 |
| 10 | 6 | 2.01 | 0.24 | 4 | −1.90 | −4.70 |
| 20 | 4 | 1.88 | 0.25 | 4 | −0.59 | −3.32 |
| | Longest Sleep Bout | | | | | |
| Dose | | Dose | | | | |
| (mg/kg PO) | N | (mg/kg PO) | SE | | | |
| 0.1 | 8 | 0.85 | 0.12 | | | |
| 0.25 | 9 | 1.49 | 0.19 | | | |
| 0.5 | 9 | 1.79 | 0.23 | | | |
| 1 | 9 | 1.89 | 0.24 | | | |
| 3 | 6 | 2.17 | 0.33 | | | |
| 10 | 6 | 2.29 | 0.36 | | | |
| 20 | 4 | 1.90 | 0.34 | | | |

Table 2. Outcome statistics:
Abbreviations: N = sample size; Adj. Mean = adjusted group mean value relative to vehicle controls; SE = standard error of the mean; LCL = lower 95% confidence limit, NREM = non-REM, i.e., all sleep other than REM sleep. The parallel reference vehicle group sample size was N = 27.
Definitions and units - means are adjusted differences from vehicle controls:
Cumulative sleep: across the first 6 hr. post-treatment, in minutes (‘Total sleep’ denotes NREM sleep + REM sleep).
Average sleep bout: average of hourly-averaged sleep bouts, across the first 6 hr. post-treatment, expressed as n-fold increase over vehicle controls.
Longest sleep bout: the longest sleep bout in the first 6 hr. post-treatment, expressed as n-fold increase over vehicle controls.
Rebound insomnia: cumulative minutes of NREM + REM sleep during the first 3 hr. of the lights on period, i.e., 7^{th}, 8^{th} and 9^{th} hours post-treatment.
REM inhibition: cumulative minutes of REM sleep during the first 12 hr. post-treatment.
Locomotor Activity Intensity (LMI): expressed as LMA counts per minute of EEG-defined wakefulness, averaged across the first 6 hr. post-treatment.

Determining efficacy. The threshold efficacy for each of the four efficacy variables is calculated by plotting the increase in each variable relative to vehicle controls during the 6 hr. period after treatment against log(dose). The threshold efficacy for each variable is that dose, estimated by 4 paramater logistic nonlinear regression, which gives the defined efficacy threshold value: +30 min. of additional accumulated non-REM sleep, +25 min. of additional accumulated total sleep, 1.75× increase in average sleep bout duration, and 1.5× increase in longest sleep bout duration. The compound of example 1 is found to have threshold efficacious doses as shown in Table 3.

TABLE 3

| | Estimated | |
|---|---|---|
| | efficacy dose | 95% confidence |
| | (mg/kg) | interval (mg/kg) |
| NREM accumulation = 30 min. | 1.5 | 0.86-2.76 |
| Total sleep accumulation = 25 min. | 0.39 | 0.27-0.58 |
| Longest sleep bout (1.75-fold increase) | 0.45 | 0.1-1.51 |
| Average sleep bout (1.5-fold increase) | 0.33 | 0.19-0.73 |

Determining undesired effects. Each ‘undesired effect’ outcome variable (see Table 2 legend for definitions), is plotted against log(dose). Negative values indicate REM inhibition, rebound insomnia and reduced LMI, respectively, in the plots below. The threshhold value for REM sleep inhibition is defined as a cumulative reduction of REM sleep of −10 min. The threshold value for rebound insomnia is defined as −20 min. The threshold value for reduced LMI is defined as −5 locomotor activity counts per minute of EEG-defined wakefulness. A significant undesired effect is defined to occur when the lower confidence limit goes below the threshold value at any dose at or below 10 times the average efficacious dose, and a dose response trend is evident for doses above the threshhold efficacy dose. For the compound of Example 1, REM sleep inhibition exceeded the threshhold value at 20 mg/kg, but this dose is more than 10 times the most conservative efficacy dose of 1.5 mg/kg (Table 3). Apparent LMI impairment at 1 mg/kg is not part of a dose related trend but is statistically significant at that dose; it is considered most likely an artifact of the small sample sizes at each dose; nevertheless, LMI impartment cannot be ruled out by these data. It is concluded that no undesired occurences of REM inhibition or rebound insomnia are observed within 10 times the most conservative efficacy dose, but locomotor activity impairment cannot be ruled out.

MK-801 induced locomotor hyperactivity in mice: The rodent MK-801 induced locomotor hyperactivity assay has been used as a predictor of anti-psychotic therapeutic potential (Corbett, R., Camacho, F., Woods, A. T., Kerman, L. L., Fishkin, R. J., Brooks, K., and Dunn, R. W. Antipsychotic agents antagonize non-competitive N-methyl-D-aspartate antagonist-induced behaviors. Psychopharmacology (1995) vol. 120: 67-74). Compounds of the invention may be evaluated for their ability to reduce locomotor hyperactivity elicited by the NMDA receptor antagonist MK-801. Studies are conducted in the dark. Locomotor activity is measured in clear Perspex™ boxes (40×40×30 cm) based on infrared fields (100×100 cm). Sixteen boxes are used, four on each field, each monitored using overhead infrared cameras (Sanyo VCV-3412P, TrackSys Ltd, UK). The cameras feed into a Quad Videoswitch (VQ403-C), TrackSys Ltd, UK), which in turn feeds a PC running the image analysis application Ethovision v8 (Noidus, NL).

All treatment tzroups have initial sample sizes of n=8. Mice are assigned to groups using a pseudorandomised block method where treatments are balanced across the time of day of testing and the test room used. On the test day, animals are transported to the testing room ante-chamber in their home cages and dosed with the test compound (0.3-3 mg/kg po) or vehicle before being placed individually into the test boxes for 1 hr., during which time locomotor activity is measured. Following this habituation period, animals are dosed with MK-801 (0.1 mg/kg SC) or vehicle as appropriate and then replaced into the test boxes where locomotor activity is measured for a further 2 hrs. The measurement of interest for LMA studies is the distance moved (cm). Unless stated otherwise, points on all graphs are plotted as the mean±standard errors.

Data are imported into STATISTICA (v9) for statistical analyses. A general linear model approach is used for all multivariate ANOVAs calculated for the data. Following ANOVA, planned comparisons are used to investigate significant differences from the control animals. Statistical significances are denoted by the following symbols: *p<0.05, **p<0.01. Mean values calculated in Statistica are imported into SigmaPlot (v11) to graph the data.

The compound of Example 1 is tested essentially as described and is found to significantly block the MK-801 induced locomotor hyperactivity. This result may be deemed as indicative of therapeutic potential as an anti-psychotic agent. (See Table 4)

TABLE 4
MK801 induced locomotor hyperactivity in Mice

| | | | Distance traveled |
|---|---|---|---|
| | | Distance traveled | after administration |
| | | during habituation | of vehicle or |
| | | with vehicle or | 0.1 mg/Kg |
| Treatment | | compound (cm) | MK-801 (cm) |
| Vehicle/Vehicle | Mean | 10516.37 | 7139.08 |
| | SEM | 826.06 | 2546.44 |
| Vehicle/MK801 | Mean | 10621.81 | 16975.47 |
| | SEM | 794.67 | 3495.07 |
| 0.3 mg/Kg Ex. 1/MK801 | Mean | 9336.73 | 8492.08 |
| | SEM | 568.32 | 2245.88 |
| 1.0 mg/Kg Ex. 1/MK801 | Mean | 8939.48 | 7258.87 |
| | SEM | 1149.26 | 1493.53 |
| 3.0 mg/Kg Ex. 1/MK801 | Mean | 7682.77 | 7085.68 |
| | SEM | 928.75 | 2372.46 |

*p<0.05, **p<0.01 v. Veh/MK801

While it is possible to administer the compounds as employed in the methods of this invention directly without any formulation, the compounds are usually administered in the form of pharmaceutical compositions comprising the compound, or a pharmaceutically acceptable salt thereof, as an active ingredient and at least one pharmaceutically acceptable carrier, diluent and/or excipient. These compositions can be administered by a variety of routes including oral, sublingual, nasal, subcutaneous, intravenous, and intramuscular. Such pharmaceutical compositions and processes for preparing them are well known in the art. See, e.g., Remington: The Science and Practice of Pharmacy (University of the Sciences in Philadelphia, ed., 21^{st} ed., Lippincott Williams & Wilkins Co., 2005).

The compositions are preferably formulated in a unit dosage form, each dosage containing from about 0.1 to about 60 mg, more usually about 1.0 to about 30 mg, as for example between about 2 and about 10 mg of the active ingredient. The term “unit dosage form” refers to physically discrete units suitable as unitary dosages for human subjects and other mammals, each unit containing a predetermined quantity of active material calculated to produce the desired therapeutic effect, in association with at least one suitable pharmaceutically acceptable carrier, diluent and/or excipient.

The compounds of Formula I are generally effective over a wide dosage range. For example, dosages per day normally fall within the range of about 0.002 to about 1.0 mg/kg, more usually from about 0.015 to 0.5 mg/kg, and as for example between 0.03 and 0.15 mg/kg of body weight. In some instances dosage levels below the lower limit of the aforesaid range may be more than adequate, while in other cases still larger doses may be employed without causing any harmful side effect, and therefore the above dosage ranges are not intended to limit the scope of the invention in any way. It will be understood that the amount of the compound actually administered will be determined by a physician, in the light of the relevant circumstances, including the condition to be treated, the chosen route of administration, the actual compound or compounds administered, the age, weight, and response of the individual patient, and the severity of the patient's symptoms.

We claim:

1. A compound of the formula 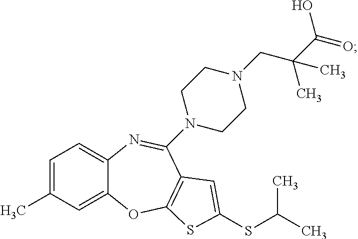 [SMILES: Cc1ccc2c(c1)Oc1sc(SC(C)C)cc1C(N1CCN(CC(C)(C)C(=O)O)CC1)=N2]
or a pharmaceutically acceptable salt thereof.

2. The compound according to claim 1 which is 2,2-dimethyl-3-(4-{8-methyl-2-[(1-methylethyl)sulfanyl]thieno[2,3-b][1,5]benzoxapezin-4-yl}piperazin-1-yl)propionic acid.

3. The compound according to claim 1 which is a mono-HCl salt.

4. The compound according to claim 1 which is a mono-phosphate salt.

5. A pharmaceutical composition comprising a compound according to claim 1, or a pharmaceutically acceptable salt thereof, and at least one pharmaceutically acceptable carrier, diluent, or excipient.

6. A method of treating psychosis associated with Alzheimer's disease comprising administering to a patient in need of such treatment an effective amount of a compound of the formula 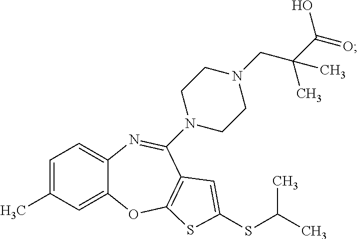 [SMILES: Cc1ccc2c(c1)Oc1sc(SC(C)C)cc1C(N1CCN(CC(C)(C)C(=O)O)CC1)=N2]
or a pharmaceutically acceptable salt thereof.

7. A method of treating agitation associated with Alzheimer's disease comprising administering to a patient in need of such treatment an effective amount of a compound of the formula 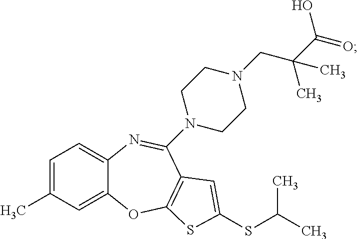 [SMILES: Cc1ccc2c(c1)Oc1sc(SC(C)C)cc1C(N1CCN(CC(C)(C)C(=O)O)CC1)=N2]
or a pharmaceutically acceptable salt thereof.

8. A method of treating aggression associated with Alzheimer's disease comprising administering to a patient in need of such treatment an effective amount of a compound of the formula 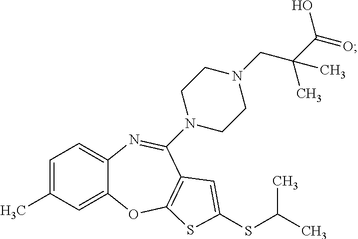 [SMILES: Cc1ccc2c(c1)Oc1sc(SC(C)C)cc1C(N1CCN(CC(C)(C)C(=O)O)CC1)=N2]
or a pharmaceutically acceptable salt thereof.

9. A method of treating psychosis associated with Parkinson's disease comprising administering to a patient in need of such treatment an effective amount of a compound of the formula 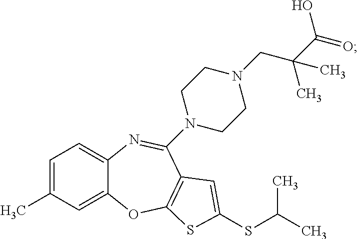 [SMILES: Cc1ccc2c(c1)Oc1sc(SC(C)C)cc1C(N1CCN(CC(C)(C)C(=O)O)CC1)=N2]
or a pharmaceutically acceptable salt thereof.

10. A method of treating agitation associated with Parkinson's disease comprising administering to a patient in need of such treatment an effective amount of a compound of the formula 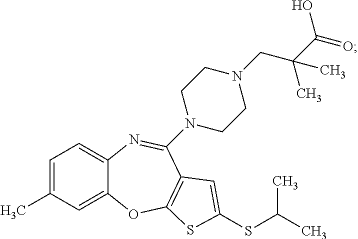 [SMILES: Cc1ccc2c(c1)Oc1sc(SC(C)C)cc1C(N1CCN(CC(C)(C)C(=O)O)CC1)=N2]
or a pharmaceutically acceptable salt thereof.

11. A method of treating aggression associated with Parkinson's disease comprising administering to a patient in need of such treatment an effective amount of a compound of the formula 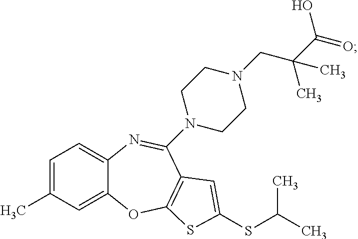 [SMILES: Cc1ccc2c(c1)Oc1sc(SC(C)C)cc1C(N1CCN(CC(C)(C)C(=O)O)CC1)=N2]
or a pharmaceutically acceptable salt thereof.
